(19) United States
(12) Patent Application Publication
(10) Pub. No.: US 2003/0212381 A1
(43) Pub. Date: Nov. 13, 2003

(54) Enteral tube feeding device with in-operation cleaning capability

(76) Inventor: Clarence Mark Whitehead III, Gulf Breeze, FL (US)

Correspondence Address: KENNETH P. GLYNN, ESQ., Glynn & Associates, P.C., 24 Mine Street, Flemington, NJ, 08822, US

(21) Appl. No.: 10/142,484

(22) Filed: May 9, 2002

(51) Int. Cl. A61M 1/06; A61M 31/00

(52) U.S. Cl. 604/514

(57) ABSTRACT

The present invention is an enteral tube feeding device having a main housing with a pump mechanism, a valving mechanism, drives and controls for pumping fluid through flexible tubing located within the main housing and functionally connected to a “Y” tubing component. The “Y” tubing component is located within the main housing with the base of the “Y” tubing component being functionally connected to the pump so as to pump fluid toward the bottom of the “Y” tubing component, the “Y” tubing component having a left upper portion and a right upper portion, each being adapted for connection to separate fluid supply sources.

### BACKGROUND OF THE INVENTION

1. Field of the Invention

The present invention relates to enteral tubes and overcoming the difficulties involved in the clogging of enteral tube feeding devices. More specifically, the present invention is a device which permits dual feed lines for alternating input to the patient as needed or periodically to flush out clogged slurry without down time of interruption of flow.

2. Information Disclosure Statement

The following patents are exemplary of prior art which relates generally to dual feeding systems for medical applications:

U.S. Pat. No. 3,860,000 to John A. Wootten and George S. Rives describes an apparatus including means for feeding a solution to the stomach of a patient, and sensing the pressure within the stomach to stop feeding when an overpressure condition exists. The stomach is then aspirated for a predetermined time. After aspiration, irrigation fluid is pulsed to the stomach.

U.S. Pat. No. 4,236,515 to Joseph N. Genese describes a gravitational flow system and equipment sets for the sequential administration of medical liquids wherein a primary liquid can be administered at a flow rate of a secondary liquid, and including a barrier substantially impervious to air to prevent the inadvertent administration of air when the secondary liquid is depleted. The sets of this invention provide parallel flow paths for the secondary liquid to insure that the flow of the primary liquid is interrupted while the secondary liquid is flowing.

U.S. Pat. No. 4,237,880 to Joseph N. Genese describes equipment which sets for the sequential administration of medical liquids wherein a primary liquid can be administered at a flow rate independent of the flow rate of a secondary liquid, and including a barrier substantially impervious to air to prevent the inadvertent administration of air when the secondary liquid is depleted. The sets of this invention employ a combined air barrier and liquid sequencing valve controlled by a common flexible membrane.

U.S. Pat. No. 4,258,717 to Jose Bisera et al. describes an apparatus for facilitating the performance of medical procedures involving the vascular system of a patient, especially in a critical care environment, such as withdrawal of blood for analysis and infusion of medication. In the taking of a blood sample, blood flows through an arterial catheter connected to an artery of the patient, past a sample station, and then through a venous catheter that brings blood back to the patient, the various mechanisms being connected by transparent plastic tubes. After a sample is taken, the tubes are flushed through the sample station, arterial catheter, and the venous catheter. Calibration of all pressure transducers requires application of high and low pressures to them, the high pressure being obtained by applying a high pressure (e.g. 200 mm Hg) to a bottle of saline solution which is also utilized to flush the tubes and which is connected to all of the transducers.

U.S. Pat. No. 4,696,671 and $, 865,584 to Paul Epstein, et al. both describe an infusion system for administering multiple infusates at individually programmable rates, volumes, and sequences in any order from any one or more of plural fluid input ports through a patient outport port and into the circulatory system of a patient. Infusates may be either continuously or time sequentially administered, and infusates may be either intermittently administered at selectively regular intervals or in time overlap to administer a dilution. Various error conditions are automatically detected and alarms generated in the event of conflicts between infusates, to identify times of no infusions, and to identify system malfunctions. The system is selectively operable, among others, in a priming mode, a maintenance mode, a normal-on mode, and a manual override mode. The system is operative to adapt actual to desired flow rates in normal operation. All fluids flow through a unitary disposable cassette without making any other system contact. Air bubbles in the fluid line are automatically detected and disposed of. Fluid pressures are monitored and system operation adjusted as a function of such pressures. Infusates may be administered from syringes as well as from standard bag or bottle containers. Infusates from a selected input port may be controllably pumped into a syringe for unsticking the syringe plunger. The system is selectively operable to adjust total fluid volume and rate to below preselected values for patients whose total fluid intake must be restricted. The system is operable to maintain an accurate record of total infusion history.

U.S. Pat. No. 4,838,856 to Patrick A. Nulreany et al. describes the flow of fluid from primary and secondary reservoirs is induced along parallel flow paths to a common infusion device under gravity or pump inducing modes, respectively. The fluid delivered to the infusion device is monitored by a flow meter to measure its actual flow rate and adjust flow along the parallel flow paths to maintain a substantially constant selected flow rate by means of a programmed control system through which selection of the flow inducing mode and the reservoir may be effected.

U.S. Pat. No. 4,941,875 to John F. Brennan describes the intravenous (I.V.) system providing for successive administration to a patient of two or more solutions but at different flow rates. Such a system includes a primary or first intravenous set including tubing which at one end thereof enters a container having a first solution at a predetermined level and which extends from that point towards the patient. The tubing of the primary set includes a check valve above the connector site. A secondary set may be temporarily or permanently connected to the connector site and includes tubing attached to a second container having a second solution at a level above the level of the first fluid. Adjustable and independent flow control devices are provided in the tubing of the primary set, one flow control device being located downstream of the connector site for controlling flow of the solution from the second container and the other flow control device being located upstream of the connector site for automatically turning on and off the first solution at its own, individually set flow rate, generally after the termination of the flow of the solution from the second container.

U.S. Pat. No. 5,069,661 to Leonard A. Trudell describes a low-pressure, low blood trauma heodynamic support system being disclosed. The system may operate as a relatively static-volume, gravity-fed, extracorporeal blood circulation and oxygenation system that consists essentially of (1) a membrane-type blood oxygenator, (2) a non-occlusive roller pump, and (3) connecting tubes. The tubes connect the system components to create a compact system capable of supporting a patient in circulatory dysfunction who is to undergo a transplant operation, and is awaiting a donor organ.

U.S. Pat. No. 5,429,485 to Larry H. Dodge describes an integral disposable manifold and pump cassette, through which fluid is pumped from inlet tubes, forming a single disposable cassette which may be inserted into a single pump housing. The integral disposable manifold and pump cassette includes first and second sheets of flexible plastic material. Sealed inlet channels are formed between the first and second sheets of flexible plastic material, with the sealed inlet channels being connectable to inlet tubes for fluid passage therethrough. The sealed inlet channels can be individually pinched off to prevent fluid passage therethrough. A sealed main channel, which is connected between the inlet channels and an outlet tube is formed between the first and second sheets of flexible plastic material. The sealed main channel includes a pumping chamber which is also formed between the first and second sheets of flexible plastic material. A flange; also formed between the first and second sheets of flexible plastic material, generally surrounds the inlet channels and the main channel. The flange facilitates the insertion and removal of the disposable manifold and pump cassette from the single pump housing. A first flexible diaphragm, extending outward from the flange, is formed in the first sheet of flexible plastic material. The first flexible diaphragm forms a portion of the pumping chamber.

Notwithstanding the prior art, the present invention is neither taught nor rendered obvious thereby.

### SUMMARY OF THE INVENTION

The present invention is an enteral tube feeding device having (a) a main housing adapted to receive a pump mechanism, a reciprocal compression valve, drives and control means; (b) a pump for pumping fluid through flexible tubing located within main housing and functionally connected to a “Y” tubing component; (c) a “Y” tubing component located within said main housing with the base of said “Y” tubing component being functionally connected to said pump so as to pump fluid toward the bottom of said “Y” tubing component, said “Y” tubing component having a left upper portion and a right upper portion, each being adapted for connection to separate fluid supply sources; (d) a reciprocal compression valve having a left compression head and a right compression head, said reciprocal compression valve being reciprocally movable from a first position to a second position and vice versa, wherein said first position, said left compression head does not compress said left upper portion of said “Y” tubing component to permit fluid to flow therethrough and said right compression head does compress said right upper portion of said “Y” tubing component to prevent fluid flow therethrough, and when said reciprocal compression valve is in said second position, said left compression head does compress said left upper portion of said “Y” tubing component to permit fluid to flow therethrough and said right compression head does not compress said right upper portion of said “Y” tubing component to prevent fluid flow therethrough; (e) drive means for driving said reciprocal compression valve from said first position to said second position and vice versa; (f) control means connected to said reciprocal compression valve drive and connected to said pump mechanism adapted to control power to said device and to control said pump and said valve drive. In other preferred embodiments, the valving mechanism is not a reciprocal compression valve, but separate valves which operate independently by the controls so that either side of the “Y” tube component may be opened or closed independent of the other side.

### BRIEF DESCRIPTION OF THE DRAWINGS

The present invention should be more fully understood when the specification herein is taken in conjunction with the drawings appended hereto wherein:

FIG. 1 illustrates a schematic diagram of the present invention enteral tube feeding device, and

FIG. 2 illustrates a front view of one preferred embodiment of the present invention device; and,

FIG. 3 shows a partial front view of another present invention enteral tube feeding device having alternative valving mechanisms.

### DETAILED DESCRIPTION OF THE PRESENT INVENTION

In the field of enteral tube feeding devices, existing machines are used to pump a slurry type solution of various nutritional and/or medicinal compositions into patients using enteral tube arrangements. The solutions may be, for example, similar to “Ensure”, although generally more viscous and more nutritious. The tubes through which these solutions and similar solutions are fed to the patient include masogastric (NG), jejunal (J-tubes) and gastric (G-tubes). These tubes often become clogged by the slurry-type solutions and usually must be replaced (sometimes surgically). This obviously entails time, expense and additional risk to the patient. To combat this, doctors write orders requiring that these tubes be disconnected periodically by the nursing staff and flushed with saline or sterile water, e.g. every 4-8 hours, as well as after the tube has been used to deliver a medication. In actual practice, doctors often forget to carry out the orders. In addition, these machines are often used in the home where family members are responsible for the care of the tube. These prior art machines have one inlet for the solution and one outlet going to the patient. The machines are controlled by a rheostat or other control mechanism to regulate pumping, so as to supply the desired milliliters per hour, generally 20/60 ml/hr. A survey mailed to 1,700 critical care nurses showed the rate of obstruction of the feeder tube by medication in patients who received 8 to 9 doses of medication per day, was 15.60. Other tests have shown tube blockage occurring in approximately 12% of the cases, with tube leakage occurring in 5% of the cases. The prior art does not address consideration for the tube clogging, the major problem in automatic feeding machines. Multiple feed systems for other medical objectives overcome some problems but create complexities which create new problems, such as operability errors, expense, replacement, down time, etc.

It is a primary object of the present invention to provide multiple feeder tubes controlled by one feeder pump, or a series of pump mechanisms if desired, through a series of solenoids or other control mechanisms which will feed solutions at a rate predetermined by a chip algorithm and timed for duration by use of a timing mechanism. The control mechanisms may include times which will have the dual purpose of turning off one fluid source so as to use the device to then feed a second solution, e.g. IV fluid, which will have the multiple purposes of cleansing the feeder tube, eliminating clogging and feeding the IV fluid to the patient. The reverse procedure may also be controlled by the timing mechanism, turning off the IV fluid and turning on the enteral feed solution. Alternatively, this may be done manually, but automatic systems are preferred. These present invention devices have the effect of reducing expense in patient care, freeing hospital personnel for more important duties and improving the comfort of the patient as the entire mechanism would need less maintenance. Further, the present invention devices offer continuous feed, eliminate shut down times and permit simple modular interchangeable Y-tube components that reduce maintenance.

Thus in one preferred embodiment of the present invention, a solid state control device, with the ability to handle multiple commands, will control the flow of multiple medical solutions, two or more, simultaneously or individually, controlling the amount of solution and the on-off function. The flow will be controlled by individual solenoids that will be turned on or off by the control device. A center valve mechanism will allow the flow of medical solutions when the appropriate solenoid is opened. When the two or more solenoids are open, the center valve mechanism will allow the total flow of all medical solutions to the patient. When the IV only solenoid is opened, the center valve mechanism will allow for the initial flooding of all tubes up to the solenoid to assure removal of all residual materials allowing for the cleansing of the inner walls of the tubes. The center valve mechanism will allow for the programmed amount of IV or other fluids at all times. The control device will have an alarm system that will trigger when any portion of the system is malfunctioning.

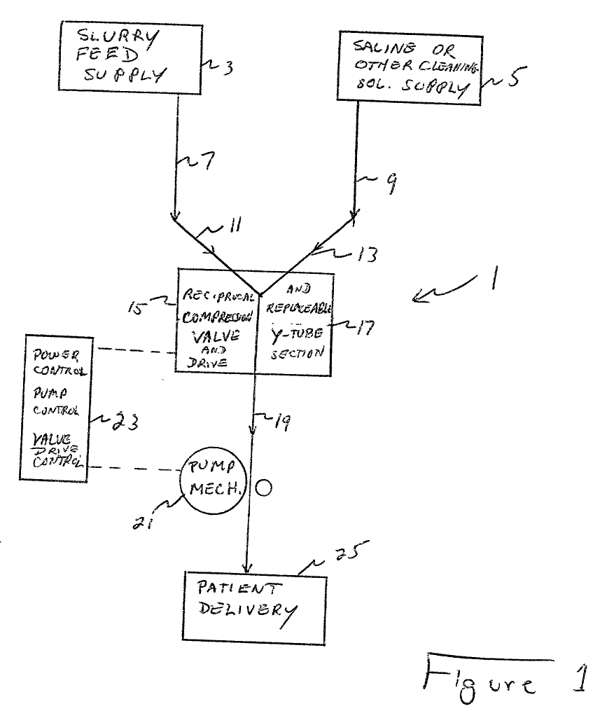

FIG. 1 shows a schematic diagram of present invention enteral tube feeding device 1, slurry feed supply 3 and saline or other cleaning solutions supply 5 are connected to gravity feed tubes 7 and 9, as shown. Gravity feed tube 7 is connected to the left upper portion 11 of a “Y” tube component and gravity feed tube 9 is connected to the right upper portion 13 of the “Y” tube component. The “Y” tube component is controlled by the reciprocal compression valve and drive 15 which operates to open one side and close the other side of the upper portions of “Y” tube component 17. At the bottom of the “Y” tube component 17 is a section 19 connected to pump mechanism 21. Control means 23 include various controls such as a power switch, a pump control and a valve drive control. These controls maintain flow to slurry feed supply 3 to patient delivery 25 and also to switch from slurry feed system 3 over to cleaning solution supply 5 to flush the tubing from the bump area to the patient delivery with little or no interruption of fluid flow. Thus, when clogging occurs flushing may be achieved without the need to shut down the system or to take apart or remove the pump. Additionally, the “Y” tubing component may be removable and replaced with new tubing as desired without disassembly of the pump or valving.

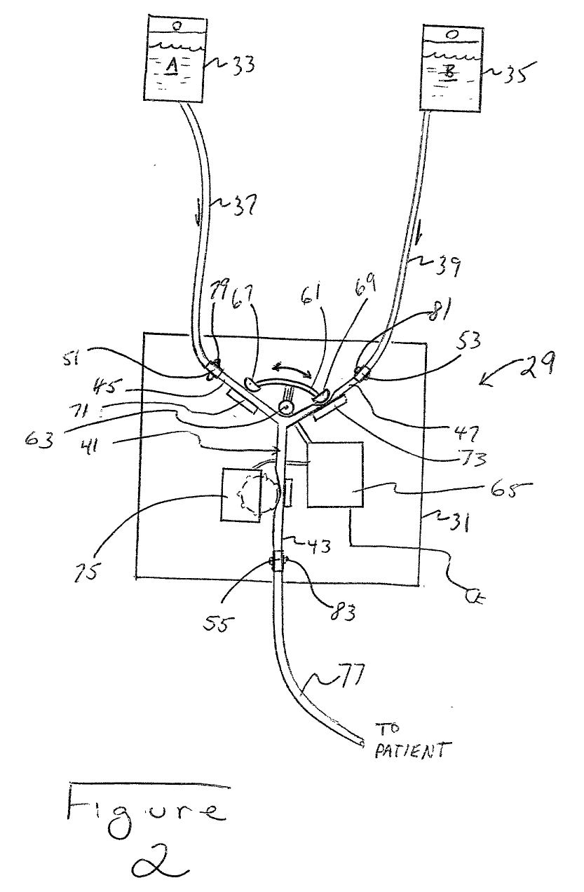

FIG. 2 shows the front view of a present invention device 29 contained within main housing 31. First fluid A is contained within gravity feed pouch 33 and second fluid B is contained within gravity feed pouch 35. Pouch 33 is connected to tube 37 for downward flow and pouch 35 is connected to flexible tube 39 for downward flow. Tube 37 is connected via connector 51 to “Y” tube component 71, and more specifically, to its left upper section 45. Tube 39 is connected via connector 33 to right upper portion 37 of “Y” tube component 41. A reciprocal compression valve 61 acts as a dual valve by reciprocating through an arc from pivot point 63 by moving from a first position (as shown) to a second position (not shown) and vice versa in accordance with the operators needs, either manually or automatically, or a combination of both. In it's first position, left compression head 67 is not compressing left upper section 45 while right compression head 69 is compressing right upper section 47, thereby permitting fluid to flow from tube 37 but not from tube 39. When pivot arm 61 swings to the left, compression head 67 closes the left upper section 45 and compression head 69 opens right upper section 47 to permit fluid B to flow and to stop the flow of fluid A. Pump 75 flows fluid down through bottom section 43 of “Y” tube component 41 to lower tube 77 to the patient. Lower tube 77 is connected via connector 55 to the bottom portion 43 of “Y” tube component 41. Connectors 51, 53 and 55 removably held in place on main housing 31 by holding means such as spring clamps 79, 81 and 83. These holding means not only position the connectors but also accurately position and hold “Y” tube component 41 in place for both attachment purposes and mechanical purposes (pumping and valving). Controlling means 65 controls power and pump speed as well as activation of the device mechanism of reciprocal compression valve 61 to permit the operator to flush the system at will or automatically at predetermined intervals without significant down time and without the need for pump replacement or disassembly.

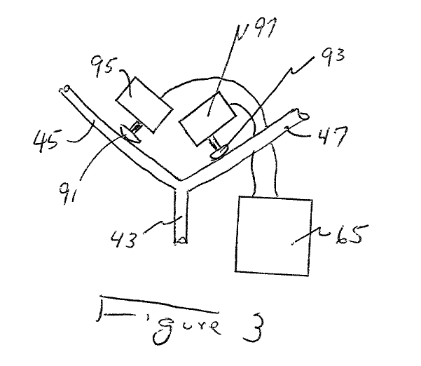

FIG. 3 shows a partial front view of the device shown in FIG. 2, except that an alternative valving mechanism is illustrated. Parts identical to FIG. 2 are identically numbered in FIG. 3. In this-embodiment, compression heads 91 and 93 are separate from one another and are controlled by solenoid switches 95 and 97 respectively. Because the compression heads 91 and 93 are separate from one another, each may be open and closed independent of one another. These solenoid switches 95 and 97 are operated independently by controlling means 65. Thus, four control possibilities exist as follows:

Both valve compression heads open;

Valve compression head 91 closed, valve compression head 93 open;

Valve compression head 91 open, valve compression head 93 closed;

Both valve compression heads closed.

By this arrangement, both of the feed supplies may be open at the same time, one may be open and the other closed or vice versa, or both may be closed. This may be automatically or manually, but is preferably done automatically by programmable control chips.

Obviously, numerous modifications and variations of the present invention are possible in light of the above teachings. It is therefore understood that within the scope of the appended claims, the invention may be practiced otherwise than as specifically described herein.

What is claimed is:

1. An enteral tube feeding device, which comprises:
(a) a main housing adapted to receive a pump mechanism, a valving mechanism, drive means and control means;
(b) a pump mechanism for pumping fluid through flexible tubing located within main housing and functionally connected to a “Y” tubing component;
(c) a “Y” tubing component located within said main housing with the case of said “Y” tubing component being functionally connected to said pump mechanism so as to pump fluid towards the bottom of said “Y” tubing component, said “Y” tubing component having a left upper portion and a right upper portion each being adapted for connection to separate fluid supply sources;
(d) a valve mechanism having a left valve with a compression head an a right valve with a compression head, and individual drive mechanisms for said left valve and said right valve, each of said left and right valves being reciprocally movable independent of the other form a first position to a second position and vice versa, wherein in said first position, a compression head does not compress an upper portion of said “Y” tubing component and permits fluid to flow therethrough and wherein in said second position, said compression head does compress said upper portion of said “Y” tubing component and prevents fluid to flow therethrough;
(e) drive means for independently driving said left valve and said right valve of said valve mechanism from said first position to said second position and vice versa;
(f) control means connected to said drive means and connected to said pump mechanism and adapted to control power to said device and to control said pump and said valve mechanism.

2. The enteral tube feeding device of claim 1 wherein said “Y” tubing component is removable and replaceable.

3. The enteral tube feeding device of claim 1 wherein said main housing also contains compression stops located on said upper portions of said “Y” tubing component opposite said compression heads.

4. The enteral tube feeding device of claim 1 further including means for switching said reciprocal compression valve from said first position to said second position and vice versa at predetermined regular intervals.

5. The enteral tube feeding device of claim 1 wherein said valve mechanism includes solenoids for switching each of said valves from said first position to said second position and vice versa.

6. The enteral tube feeding device of claim 2 wherein the enteral tube feeding device of claim 1 wherein said valve mechanism includes solenoids for switching each of said valves from said first position to said second position and vice versa.

7. The enteral tube feeding device of claim 1 wherein said main housing further includes removably attached connectors at each of three open ends of said “Y” tube components for exact positioning and holding of said “Y” tube component.

8. The enteral tube feeding device of claim 1 wherein said pump mechanism includes at least one peristaltic pump.

9. The enteral tube feeding device of claim 1 wherein said control means include manual control means.

10. The enteral tube feeding device of claim 1 wherein said control means include automatic programmable control means.

11. The enteral tube feeding device, which comprises:
(a) a main housing adapted to receive a pump mechanism, a reciprocal compression valve, drive means and control means;
(b) a pump mechanism for pumping fluid through flexible tubing located within main housing and functionally connected to a “Y” tubing component;
(c) a “Y” tubing component located within said main housing with the base of said “Y” tubing component being functionally connected to said pump fluid towards the bottom of said “Y” tubing component, said “Y” tubing component having a left upper portion and a right upper portion, each being adapted for connection to separate fluid supply sources;
(d) a reciprocal compression valve having a left compression head and a right compression head, said reciprocal compression valve being reciprocally movable from a first position to a second position and vice versa, wherein when said reciprocal compression valve is in said first position, said left compression head does not compress said left upper portion of said “Y” tubing component and permits fluid to flow therethrough and said right compression head does compress said right upper portion of said “Y” tubing component to prevent fluid flow therethrough, and when said reciprocal compression valve is in said second position, said left compression head does compress said left upper portion of said “Y” tubing component to prevent fluid to flow therethrough and said right compression head does not compress said right upper portion of said “Y” tubing component to permit fluid flow therethrough;
(e) drive means for driving said reciprocal compression valve from said first position to said second position and vice versa;
(f) control means connected to said reciprocal compression valve drive and connected to said pump mechanism and adapted to control power to said device and to control said pump and said valve drive.

12. The enteral tube feeding device of claim 11 wherein said “Y” tubing component is removable and replaceable.

13. The enteral tube feeding device of claim 11 wherein said main housing also contains compression stops located on said upper portions of said “Y” tubing component opposite said compression heads.

14. The enteral tube feeding device of claim 11 further including means for switching said reciprocal compression valve from said first position to said second position and vice versa at predetermined regular intervals.

15. The enteral tube feeding device of claim 11 wherein said main housing further includes removably attached connectors at each of three open ends of said “Y” tube components for exact positioning and holding of said “Y” tube component.

16. The enteral tube feeding device of claim 12 wherein said main housing further includes removably attached connectors at each of three open ends of said “Y” tube components for exact positioning and holding of said “Y” tube component.

17. The enteral tube feeding device of claim 11 wherein said pump mechanism includes at least one peristaltic pump.

18. The enteral tube feeding device of claim 11 wherein said control means include manual control means.

19. The enteral tube feeding device of claim 11 wherein said control means include automatic programmable control means.

20. The enteral tube feeding device of claim 11 wherein said control means include both manual and automatic programmable control means.
